(19) United States
(12) Patent Application Publication
(10) Pub. No.: US 2021/0169937 A1
(43) Pub. Date: Jun. 10, 2021

(54) METHOD FOR THE TREATMENT OF A TUMOR PATIENT WITH ADOPTIVE T CELL IMMUNOTHERAPY

(71) Applicant: NORDWEST POLYBIOCEPT BIOSCIENCE GMBH, Frankfurt am Main (DE)

(72) Inventors: Ernest DODOO, Frankfurt am Main (DE); Elke JÄGER, Frankfurt am Main (DE); Julia KARBACH, Frankfurt am Main (DE); Markus MAURER, Frankfurt am Main (DE)

(73) Assignee: NORDWEST POLYBIOCEPT BIOSCIENCE GMBH, Frankfurt am Main (DE)

(21) Appl. No.: 17/263,350

(22) PCT Filed: Jul. 26, 2019
(86) PCT No.: PCT/EP2019/070283

§ 371 Date: Jan. 26, 2021

(30) Foreign Application Priority Data
Jul. 27, 2018 (EP) 18186026.3

(51) Int. Cl. A61K 35/17; A61K 31/519; A61K 31/436; A61K 31/675; A61K 31/52; A61K 38/20; A61K 39/395; A61K 38/17; A61P 35/00; C12N 5/0783

(52) U.S. Cl. CPC A61K 35/17; A61K 31/519; A61K 31/436; A61K 31/675; A61K 31/52; A61K 38/2013; C12N 2501/25; A61K 38/1793; A61P 35/00; C12N 5/0636; C12N 2501/2302; C12N 2501/2315; C12N 2501/2321; A61K 39/3955

(57) ABSTRACT

The present invention relates to a method of treating a tumor disease, comprising one or more administrations of a T cell product, a T cell product for use in a method of treating a tumor disease, as well as a kit for use in a method of treating a tumor disease.

### FIELD OF THE INVENTION

The present invention relates to a method of treating a tumor disease, a T cell product for use in said method of treating a tumor disease as well as a kit for use in the method of treating a tumor disease.

### BACKGROUND OF THE INVENTION

Adoptive cell therapy (ACT) with T cells is one of the most promising advances for the treatment of tumor diseases. ACT has produced remarkable results in the treatment of individual patients with tumors in the last decades. In this type of cell therapy, the patient's immune system is stimulated with the intent of promoting an antigen specific anti-tumor effect using the body's own immune cells.

Clinically relevant and long-term remissions have been achieved in patients with melanoma using T cells directed against tumors (tumor reactive T cells)^{1,2}. These approaches usually rely on the harvesting of T cells from peripheral blood or tumor infiltrating lymphocytes (TILs) from tumor lesions.

TIL therapy has shown clinical benefit for patients with chemotherapy-refractory cancer, such as metastatic melanoma, cholangiocarcinoma, renal cell carcinoma, colorectal, cervical, as well as ovarian cancer¹⁻³. Clinical efficacy of TIL in patients with solid tumors has been ascribed to the joint contribution of the recognition of multiple individual neoantigens as well as shared tumor antigens (TAAs) associated with enhanced tissue homing capacity and strong immune effector functions against tumor tissue.

Glioblastoma multiforme (GBM) is a high-grade central nervous system (CNS) tumor, with a poor 5-year survival rate of under 5%⁴. Although several improved surgical strategies like temozolomide-based chemotherapy, adjunctive therapy with bevacizumab (anti-vascular endothelial growth factor, VEGF antibody) and radiotherapy are available, the clinical outcome for GBM remains dismal⁴. In approximately 95% of patients, tumor recurrence occurs and complete remissions at this stage are rare exceptions. Innovative treatment strategies and regimens encompassing microparticles, molecular therapeutics and repurposed drugs for GBM have been described recently⁵⁻⁹. However, these strategies have not been validated in clinical trials yet.

Existing clinical evidence advocates for further evaluation of TIL therapy to treat GBM. In 1999, a pilot study showed that intrathecal re-infusion of TIL combined with interleukin (IL)-2 was safe and resulted in clinical responses in 5/6 patients with recurrent GBM¹⁰. More recently, complete tumor regression was observed after a total of six cycles of intracavitary delivery of IL-13 receptor alpha 2 (IL-13Ra2)-specific chimeric antigen receptor (CAR) T cells to a patient with recurrent GBM¹¹.

As reported previously, GBM-derived TILs can be successfully isolated and effectively propagated in vitro with a combination of the gamma-chain cytokines IL-2, IL-15 and IL-21; such TILs exhibit antigen-specific pro-inflammatory and cytotoxic anti-tumor functions coupled with a central memory phenotype^{12,13}.

Anti-tumor functions, i.e. strong cytotoxicity, has been described for CD8+ and CD4+ TILs (reviewed by Zanetti, 2015)¹⁴. Furthermore, also clinical relevant (anti-tumor) responses can be assigned to central memory T cells, defined by CD45RA-CCR7+. The phenotype of such T cells can be determined by using the ex vivo expanded T cell population, as well as by host factors after adoptive transfer.

In WO 2015/189357 A1 the combination of IL-2, IL-15 and IL-21 has been described for the expansion of lymphocytes, in particular T cells. T cell populations obtained by expansion in the presence of the cytokines are able to not only recognize autologous tumor calls but also kill such tumor cells in vitro. Additionally, WO 2015/189357 A1 also describes a variety of T cell products obtained from an expansion of T cells from the tumor, i.e. TILs, or peripheral blood of patients with pancreatic cancer or glioblastoma. With the expansion protocol, using a cytokine cocktail containing IL-2, IL-15 and IL-21, it is possible to produce several T cell products in parallel. These T cell products in general show a phenotype distribution advantageous for the active immunotherapy.

Tran et al. (2015) describe that TILs from 9/10 patients with metastatic gastrointestinal cancers, which were expanded in the presence of IL-2, contained CD4+ and/or CD8+ T cells that recognized one to three neo-epitopes derived from somatic mutations expressed by the patient's own tumor¹⁵.

In 2014, Tran et al. described an immunotherapy using mainly CD4+ T cells for treating epithelial cancer¹⁶. After which therapy the patient achieved a decrease in target lesions with prolonged stabilization of disease. Two years later, the same group identified a polyclonal CD8+ T cell response against mutant KRAS in cancer in TILs obtained from a patient with metastatic colorectal cancer². Interestingly, objective regression of all lung metastases has been observed in this study, after the infusion of 1.48×10¹¹ TILs, which were expanded in the presence of IL-2 and which consisted of approximately 1.11×10¹¹ CD8+ T cells reactive to the mutated KRAS.

In both approaches, the patients received a non-myeloablative lymphodepletion chemotherapy regime before TIL administration. This lymphodepleting regimen consists of a treatment with cyclophosphamide at a dose of 60 mg/kg body weight for 2 days, followed by fludarabine treatment of 25 mg per square meter of body-surface area for another 5 days. After the lymphodepletion, the patient received a single infusion of TILs, which was followed by the administration of either four or five doses of IL-2 (720 000 IU/kg). As reviewed in Rosenberg and Restifo (2015), this lymphodepleting preparative regimen described is the most frequently used regimen applied in ACTs these daysl.

However, the elevated concentrations of immunosuppressant and cytostatic agents applied prior to TIL administrations can cause severe side effects in the already weakened patient. Accordingly, there is a need in the art for improved methods for treating tumor diseases using ACT.

Thus, it is an object of the present invention to improve and further develop ACTs for treatment of tumor diseases.

### SUMMARY OF THE INVENTION

This object is solved by the subject matter of the present invention. The inventors have identified that one or more administrations of a T cell product in combination with a preceding lymphodepletion comprising less than two treatments with an immunosuppressant in a method of treating a tumor disease results in complete tumor regression.

Thus, in a first aspect the invention provides a method of treating a tumor disease, comprising one or more administrations of a T cell product, wherein at least one administration is preceded by a lymphodepletion, wherein said lymphodepletion comprises less than two immunosuppressant treatments.

According to a second aspect, the invention provides a T cell product for use in said method of treating a tumor disease, wherein the method comprises one or more administrations of a T cell product, wherein at least one administration is preceded by a lymphodepletion, wherein said lymphodepletion comprises less than two immunosuppressant treatments.

Furthermore, according to a third aspect, the invention provides a kit for use in a method of treating a tumor disease, wherein the method comprises one or more administrations of a T cell product, wherein at least one administration is preceded by a lymphodepletion, wherein said lymphodepletion comprises less than two immunosuppressant treatments.

### FIGURES

FIG. 1 shows a treatment schedule indicating TIL infusion and cyclophosphamide administration (A). A lymphodepletion using 60 mg/kg cyclophosphamide (CTX) was performed one day prior to TIL administration on either occasion (TIL-A and TIL-B). Eight hours after TIL administration, IL-2 (60,000 IU/kg) was administered i.v., followed by anti-sTNF-αR (25 mg) s.c. and anti-IL-6R (4 mg/kg) i.v. 24 and 72 hours after TIL-A and TIL-B infusion, respectively. Representative histopathological analysis are shown in (B). HE stainings of the resected tumors before (day −43), post TIL-A (day 1), and post TIL-B (day 15) treatment shows the necrotic transformation of the tumor mass after the second TIL treatment.

FIG. 2 shows representative MRI and CT scans illustrating GBM regression. The MRI on Day −43, before partial resection: T2, DWI and. MRI Day −1 pre-TIL-A infusion: T2, DWI, ADC, Flair and T1 after contrast administration. CT scan after contrast agent administration and enhancement (contrast enhancement) at Day+1 post-TIL-A infusion. The MRI Day+2 post-TIL-A infusion (ADC with T2 shine through). MRI on Day+6 post-TIL-A infusion) DWI and ADC (Day+6 post-TIL-A infusion). MRI on Day+10, i.e. Day −1 of TIL-B infusion: T2 and ADC with T2 shine through, MRI (T2) on Day+10 post-TIL-B infusion as well as Day+24 post-TIL-A infusion. Key: DWI=diffusion-weighted imaging MRI; ADC=apparent diffusion coefficient MRI; CE-CT=contrast-enhanced CT.

FIG. 3 shows a flow cytometric analysis of T cell phenotypes (left panel) and CD107a induction (right panel) in TIL after exposure to PMA expressed as percentage of CD107a—positive T cells, the dotted lines indicate the constitutive CD107a expression.

FIG. 4 shows the T-cell receptor (TCR) V beta (Vβ) repertoire of the TIL cell products. The major vβ families present in the TIL cell products are highlighted.

FIG. 5 shows the results of anti-tumor activity analyses. (A) IFN-γ production in TIL after 24-hours stimulation with OKT3 to gauge TIL functionality prior to infusion. (B) Cytotoxic potential of the TIL cell products in a standard Chromium-51 release assay, the lysis of chromium-51 (Cr51)-labeled target cells (autologous tumor cell line as well as the control leukemia cell line K562) is illustrated. Allogeneic GBM cell lines U-373 (ATCC no: HTB-17) and DBTRGO5 (ATCC no: CRL-2020), Daudi B-lymphoma cell line and the autologous EBV-transformed B cell line served as controls.

FIG. 6 shows the results of a cold target inhibition assays. (A) Results of the assay using a constant E:T ratio of 90:1, Highest blocking was commensurate in the presence of higher numbers of cold tumor cells (up to almost 100% at 90:1 and approximately 95% at 30:1) co-incubated with hot tumor cells. (B) A set ratio of cold:hot tumor cell was used (90:1) to gauge TIL activity at varying cell numbers of T cells. Highest TIL activity was observed when a greater number of T cells were present in the control co-culture with the autologous tumor cell line (ATCL) alone and dropped in a dose-dependent manner. Conversely, no TIL activity could be observed when the cold ATCL had been pre-incubated with the hot ATCL prior to the target inhibition assay.

### DETAILED DESCRIPTION OF THE INVENTION

### Definitions

The term “tumor disease” according to the invention refers to a type of abnormal and excessive growth of tissue. The term as used herein includes primary tumors and secondary tumors as well as metastasis.

A “primary tumor” according to the present application is a tumor growing at the anatomical site where tumor progression began and proceeded to yield a cancerous mass.

A “metastasis” according to the invention refers to tumors that develop at their primary site but then metastasize or spread to other parts of the body. These further tumors are also called “secondary tumors”.

As used herein an “antigen” is any structural substance, which serves as a target for the receptors of an adaptive immune response, T-cell receptor, or antibody, respectively. Antigens are in particular proteins, polysaccharides, lipids, and substructures thereof such as peptides. Lipids and nucleic acids are in particular antigenic when combined with proteins or polysaccharides.

“Disease associated antigens” are antigens involved in a disease. Accordingly, clinically relevant antigens can be tumor-associated antigens (TAA).

“Tumor associated antigens” or “TAA” according to the invention are antigens that are presented by MHC I or MHC II molecules or non-classical MHC molecules on the surface of tumor cells. As used herein TAA includes “tumor-specific antigens”, which are found only on the surface of tumor cells, but not on the surface of normal cells.

“Expansion” or “clonal expansion” as used herein means production of daughter cells all arising originally from a single cell. In a clonal expansion of T cells, all progeny share the same antigen specificity.

In agreement with the general understanding in the art “T cell” or “T lymphocyte”, is a type of lymphocyte (a subtype of white blood cell) that plays a central role in cell-mediated immunity. T cells can be distinguished from other lymphocytes, such as B cells and natural killer cells, by the presence of a T-cell receptor on the cell surface. They are called T cells because they mature in the thymus from thymocytes.

Genetically modified T cells (GM T cells) are in particular T cells that have been genetically modified to alter the T-cell specificity. The GM T cells may be generated through the expression of specific TCR α and β chains, which mediate the antigen-recognition process²². The GM T cells may be obtained by immunising transgenic mice that express the human leukocyte antigen system with human tumour proteins to generate T cells expressing TCRs against human antigens. An alternative approach is allogeneic TCR gene transfer, in which tumour-specific T cells are isolated from a patient experiencing tumour remission and the reactive TCR sequences are transferred to T cells from another patient who shares the disease but is non-responsive. Finally, in vitro technologies can be employed to alter the sequence of the TCR, enhancing their tumour-killing activity by increasing the strength of the interaction (avidity) of a weakly reactive tumour-specific TCR with target antigen²². A specific group of genetically engineered T cells are CAR T cells. CARs combine both antibody-like recognition with T-cell-activating function. They are composed of an antigen-binding region, typically derived from an antibody, a transmembrane domain to anchor the CAR to the T cell, and one or more intracellular signalling domains that induce persistence, trafficking and effector functions in transduced T cells. Sequences used to define the antigen-targeting motif for a CAR are typically derived from a monoclonal antibody, but ligands and other receptors can also be used²².

“PBMCs” as used herein refers to peripheral blood mononuclear cells, which can be obtained from peripheral blood. PBMCs mainly consist of lymphocytes, i.e. T cells, B cells, and NK cells, and monocytes. “PBMCs” also relate to predecessor peripheral blood mononuclear cell. A PBMC, which is turned into a GM T cell, is also referred to as genetically engineered PBMC.

“TIL” according to the invention refers to tumor infiltrating lymphocytes. These are lymphocytes, in particular T cells predominantly found in a tumor. A lymphocyte sample derived from tumor is also referred as TIL. TIL also relates to any kind of lymphocyte that is located in, on or around a tumor or to lymphocytes that have contacted tumor tissue or tumor cells, respectively. TIL also relate to predecessor TILs. A TIL, which is turned into a GM T cell, is also referred to as genetically engineered TIL.

A “T cell product” as used herein refers to a population of T cells for use in immunotherapy. The “T cell product” can be obtained by (clonal) expansion of T cells or GM T cells. The T cells can be autologous, allogeneic, or genetically modified T cells.

A “TIL cell product” is a T cell product, which is obtained by clonal expansion of TIL or GM TIL.

As used herein, the terms “regulatory T cells” or “Tregs” refer to a subpopulation of T cells that modulate the immune system in that they suppress immune responses of other cells. Tregs tend to be upregulated in individuals with a tumor disease, and they seem to be recruited to the site of many tumors. Tregs are thought to suppress tumor immunity, thus hindering the body's innate ability to control the growth of cancerous cells.

The terms “preceding”, “preceded”, “is preceded by” as used according to the invention refers to a single method step that is performed before another mentioned method step at a certain time point or within a certain time interval. This time point or time interval can be from less than one hour to up to several month. The term refers either to different steps or to steps of the same type. Importantly, the term does not exclude that between steps of the same type no different step can be performed.

The terms “followed” or “following” or “is followed by” as used herein refers to a timely separated but subsequent step or event.

The term “lymphodepletion” as used herein refers to the destruction and/or ablation of lymphocytes and T cells in the patient, prior to immunotherapy. Accordingly, the lymphodepletion leads to successive reduction of immune cells, which is called lymphopenia. Another, no mutually exclusive effect of the lymphodepletion is the reduction of Tregs.

The term “immunosuppressant” refers to drugs that suppress, inhibit, or prevent activity of the immune system. As used in the present invention the term “immunosuppressant” refers to drugs typically administered in chemotherapy prior to ACT. In chemotherapy the “immunosuppressant” eliminates Tregs in naive and tumor-bearing hosts, induces T cell growth factors, such as type I IFNs, and/or enhances grafting of adoptively transferred, tumor-reactive effector T cells by the creation of an immunologic space niche.

The term “autologous” means that both the donor and the recipient are the same person. The term “allogenic” means that the donor and the recipient are different persons.

As used herein, “interleukin 2” or “IL-2” refers to human IL-2 and functional equivalents thereof. Functional equivalents of IL-2 include relevant substructures or fusion proteins of IL-2 that remain the functions of IL-2. Similarly, “interleukin 15” or “IL-15” refer to human IL-15 and functional equivalents thereof. Functional equivalents of IL-15 include relevant substructures or fusion proteins of IL-15 that remain the functions of IL-15. “Interleukin 21” or “IL-21” refer to human IL-21 and functional equivalents thereof. Functional equivalents of IL-21 include relevant substructures or fusion proteins of IL-21 that remain the functions of IL-21.

The term “anti-IL-6R” as used herein refers to an anti-IL-6 receptor antibody and functional variants thereof, directed to human interleukin 6 (IL-6)-receptors. Functional variants of anti-IL-6R include relevant substructures or fusion proteins of anti-IL-6R that remain the functions of anti-IL-6R. Anti-IL-6R is commercially available as, for example, tocilizumab or atlizumab.

The term “sTNF-αR” as used herein refers to human soluble tumor necrosis factor-a receptors and functional variants thereof. There are two native receptor subtypes known in the art, namely TNFR superfamily member 1A (TNFR 1; UniProt P19438) and TNFR superfamily member 1B (TNFR 2; UniProt P20333). Functional variants of sTNF-αR include relevant substructures or fusion proteins of sTNF-αR that remain the functions of sTNF-αR. Genetically modified sTNF-αR is commercially available as, for example, etanercept or benepali.

“Clinical/biological relevance” as used herein relates to the ability of a T cell to provide at least one of the following: containment of tumor cells, destruction of tumor cells, prevention of metastasis, stop of proliferation, stop of cellular activity, stop of progress of cells to malignant transformation, prevention of metastases and/or tumor relapse, including reprogramming of malignant cells to their non-malignant state; prevention and/or stop of negative clinical factors associated with cancer, such as malnourishment or immune suppression, stop of accumulation of mutations leading to immune escape and disease progression, including epigenetic changes, induction of long-term immune memory preventing spread of the disease or future malignant transformation affecting the target (potential tumor cells), including connective tissue and non-transformed cells that would favor tumor disease.

The transitional term “comprising”, which is synonymous with “including,” “containing,” or “characterized by,” is inclusive or open-ended and does not exclude additional, unlisted elements or method steps.

Method of Treatment

According to a first aspect, the invention provides a method of treating a tumor disease, which method comprises one or more administrations of a T cell product, wherein at least one administration is preceded by a lymphodepletion, wherein said lymphodepletion comprises less than two immunosuppressant treatments.

The method has several advantages over the already existing methods. In the present method, the lymphodepletion comprises less than two immunosuppressant treatments, which is relatively “mild” compared to the standard treatments, which means that the lymphodepletion used before T cell administration does not completely shut down the patient's immune system and thus lower the risk of side-effects known from the methods of the prior art. Consequently, the method according to the invention can significantly improve the patient's conditions during chemotherapy before T cell administration.

In example 2, tumor regression was observed already after a single administration of a T cell product and a second T cell product administration eliminates the patient's tumor tissue completely. Since the method according to the invention is carried out under relatively mild conditions, i.e. a reduced lymphodepleting regimen and/or relatively low cell numbers that are infused into the patient, it is possible to carry out a plurality of administrations of a T cell product. Thus the number of administrations in the method according to the invention may be, for example, one administration, two administrations, three administrations, four administrations, or five administrations. A plurality of administrations can support and/or enhance the positive outcome of the method as it is shown for two consecutive administrations in example 2. Accordingly, in a preferred embodiment of the invention the method comprises at least two administrations of a T cell product. In another embodiment of the invention, the method comprises at least three administrations of a T cell product. In a further embodiment of the invention, the method comprises at least four administrations of a T cell product.

Most tumors elicit an immune response in the host that is mediated by tumor antigens, thus distinguishing the tumor from other non-cancerous cells. This causes large numbers of TILs to be found in the tumor microenvironment targeting cancerous cells and therefore slow down or terminate the development of the tumor. However, this process is complicated because Tregs preferentially traffic to the tumor microenvironment. While Tregs normally make up only about 4% of CD4+T cells, they can make up as much as 20-30% of the total CD4+ population around the tumor microenvironment.

High levels of Tregs in the tumor microenvironment are associated with poor prognosis in many cancers. This indicates that Tregs suppress TILs and hinder the body's immune response against the tumor.

Therefore, in ACT, a preparatory lymphodepleting regimen is established before the T cell product is administered. The goal of this lymphodepletion is in general to decrease the amount of circulating Tregs in the patient. In this regard, it is advantageous to introduce no additional Tregs with the T cell product, i.e. Tregs that have not faced any immunosuppressant treatment.

The inventors have identified, that good anti-tumor activity is achieved when the amount of Tregs in the T cell product administered in the above method is below 2.5%, preferably below 1.5%. Best results are achieved when the amount of Tregs in the T cell product is below 0.5% or 0.1%. Ideally, the T cell product does not contain any Tregs.

Accordingly, in one embodiment the content of regulatory T cells (Tregs) within the T cell product is below 2.5%. The content of Tregs may be, for example 0.01%, 0.03%, 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1.0%, 1.05%, 1.1%, 1.15%, 1.2%, 1.25%, 1.3%, 1.35%, 1.4%, 1.45%, 1.5%, 1.55%, 1.6%, 1.65%, 1.7%, 1.75%, 1.8%, 1.85%, 1.9%, 1.95%, 2.0%, 2.05%, 2.1%, 2.15%, 2.2%, 2.25%, 2.3%, 2.35%, 2.4%, or 2.45%.

In an embodiment of the invention, the content of Tregs within the T cell product is below 1.5%. Preferably, the content of Tregs within the T cell product is below 0.5%. In another embodiment of the invention, the content of Tregs within the T cell product is below 0.1%.

As T cells, in particular TILs, can be directed to different types of tumor, i.e. tumors from which environment they have been isolated, the method is suitable for the treatment of a variety of tumor diseases such as brain cancer, pancreas cancer, tumors derived from the neural crest, e.g. neuroblastoma, ganglioneuroma, ganglioneuroblastoma, and pheochromocytoma, epithelial, e.g. skin, colon, or breast, and mesenchymal origin, e.g. adipocytic, cartilaginous, fibrous, fibroblastic, myofibroblastic, osseous, or vascular, as well as hematopoietic tumors, e.g. blood, bone marrow, lymph, or lymphatic system.

According to an embodiment of the invention, the tumor disease is selected from brain cancer, pancreas cancer, hematopoietic tumors, tumors derived from the neural crest, and tumors of epithelial or mesenchymal origin.

In one embodiment of the invention, the tumor disease is brain cancer. Preferably, the tumor disease is an astrocytoma. More preferably, the tumor disease is GBM. As shown in example 2, GBM can be successfully treated by the method according to the invention comprising administrations of a TIL cell product. Accordingly, in a preferred embodiment of the invention, the T cell product is a TIL cell product.

A common side effect of many immunosuppressive drugs is immunodeficiency, because the majority of them act non-selectively, resulting in increased susceptibility to infections and decreased cancer immunosurveillance. Administration of immunosuppressive drugs in particularly high doses or over long periods of time may even require stem cell transplantation, because the chemotherapy can completely destroy bone marrow. There are also other side effects, such as hypertension, dyslipidemia, hyperglycemia, peptic ulcers, lipodystrophy, moon face, liver, and kidney injury. The immunosuppressive drugs also interact with other medicines and affect their metabolism and action. Actual or suspected immunosuppressive agents can be evaluated in terms of their effects on lymphocyte subpopulations in tissues using immunohistochemistry.

The preparatory lymphodepleting regimen used in the method according to the invention differs significantly from that employed in other known T cell studies, where the same dose of immunosuppressant is used twice on days 1 and 2, followed by fludarabine at 25 mg/m² on days 3-7 preceding T cell infusion. This high-dose conditioning perpetrates significant lymphopenia. The conditioning regimen with a single immunosuppressant dose given on day −1 according to the invention mediated mild lymphopenia and moderate neutropenia but does not cause complete lymphodepletion in the patient and thus reduces the risk of potential side effects.

Thus, in a further embodiment of the invention, each T cell administration is preceded by a lymphodepletion, wherein each lymphodepletion comprises less than two immunosuppressant treatments. In a preferred embodiment of the invention, each lymphodepletion comprises one immunosuppressant treatment.

To further lower the risk of unwanted side effects, the total concentration of the immunosuppressant in one lymphodepletion or the concentration of the immunosuppressant per treatment can be reduced by the method according to the invention.

Sufficient lymphodepletion and reduction of Tregs have been observed when the immunosuppressant is used in a total concentration of up to 65 mg/kg in each lymphodepletion. In addition, the treatment with up to 80 mg/kg is a sufficient reduction of the total immunosuppressant concentration per lymphodepletion compared to known methods.

Accordingly, in one embodiment of the invention the total concentration of the immunosuppressant in each lymphodepletion is up to 80 mg/kg. The total concentration of the immunosuppressant in each lymphodepletion may be, for example, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, or 80 mg/kg. Preferably, the total concentration of the immunosuppressant in each lymphodepletion is up to 75 mg/kg. More preferably, the total concentration of the immunosuppressant in each lymphodepletion is up to 70 mg/kg. Most preferably, the total concentration of the immunosuppressant in each lymphodepletion is up to 65 mg/kg.

Moreover, sufficient lymphodepletion and Treg reduction can be achieved when a lymphodepleting regimen is established by treatment with the immunosuppressant already at low concentrations of from 5 mg/kg per treatment to high concentrations of 80 mg/kg. Sufficient lymphodepletion together with good drug compatibility is achieved when concentrations are applied from 20 mg/kg to 65 mg/kg per treatment.

Thus, in another embodiment of the invention the concentration of the immunosuppressant in each treatment is in a range of from 5 mg/kg to 80 mg/kg.

The concentration of the immunosuppressant in each treatment may be for example, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, or 80 mg/kg. Preferably, the concentration of the immunosuppressant in each treatment is in a range of from 10 mg/kg to 75 mg/kg. More preferably, the concentration of the immunosuppressant in each treatment is in a range of from 15 mg/kg to 70 mg/kg. Most preferably, the concentration of the immunosuppressant in each treatment is in a range of from about 20 mg/kg to about 65 mg/kg.

Generally, the immunosuppressant that can be used in the present invention is selected from drugs that induce lymphopenia without significantly affecting hematopoietic stem cells and that reduce immune-suppressant and tumor-promoting activities, such as, for example, the production and/or activity of IL-10 and/or TGF-β.

As the administration of the T cell product according to the invention does not introduce substantive amounts of new Tregs into the patient, it is advantageous to select an immunosuppressant that also lowers the number of Tregs in the patient. Such an immunosuppressant may be a cytostatic drug. An additional advantage of the use of a cytostatic drug as an immunosuppressant is that it can be administered in lower dosage compared to other frequently used immunosuppressant.

Thus, in one embodiment of the invention the immunosuppressant is a cytostatic drug, preferably the immunosuppressant is selected from the group consisting of cyclophosphamide, azathioprine, methotrexate, and rapamycin.

An additional advantage of the present invention is that the method is highly variable regarding the number of cells to be administered. For example, in a stable patient, a T cell product with a high number of cells may be given as an initial dose. A high number of cells may be, for example, 10¹¹, 5×10¹⁰, or 10¹⁰ cells. When the condition worsens, a second administration of a T cell product with a lower cell number may be given.

Conversely, for a patient in poor condition, the risk of overloading the organism with immune cells can be reduced by first administering a T cell product with a low number of cells. A low number of cells may be, for example, 10⁷, 5×10⁷, 10⁸, 5×10⁸, or 10⁹ cells. If the patient tolerated the first administration well and/or the patient's conditions improve after the first administration and/or the overall therapy is effective, a further administration of a T cell product with a higher cell number can be performed.

Accordingly, in one embodiment of the invention the number of cells in the T cell product in one administration is higher than the number of cells in the T cell product of the preceding administration.

In another embodiment of the invention, the number of cells in the T cell product in one administration is lower than the number of cells in the T cell product of the preceding administration.

Furthermore, utilizing lower cell numbers compared to previous studies favoring the technical feasibility of faster T cell production combined with anti-tumor activity. Tumor regression is observed until a lower limit of about 10⁸ cells in the T cell product is used. Tumor regression is observed for T cell products containing 10⁸ to 10¹¹ cells. The cell number may be, for example, 10⁸, 5×10⁸, 10⁹, 5×10⁹, 10¹⁰, 5×10¹⁰, or 10¹¹ cells. Good results are achieved when 10⁸ to 10¹⁰ cells are present in the T cell product. Best results are observed with cell numbers from 10⁸ to 10⁹ cells.

Thus, in one embodiment of the invention, the T cell product comprises a cell number of from 10⁸ to 10¹¹ cells. The cell number may be, for example, 10⁸, 5×10⁸, 10⁹, 5×10⁹, 10¹⁰, 5×10¹⁰, or 10¹¹ cells. Preferably, the T cell product comprises a cell number of from 10⁸ to 10¹⁰ cells. More preferably, the T cell product comprises a cell number of from 10⁸ to 10⁹ cells. Most preferably, the T cell product comprises a cell number of about 10⁸ cells.

It has been observed that supporting the T cell administration with a single dose of IL-2 results in elevated anti-tumor activity and rapid expansion of the T cells in the patient.

Without wanting to be bound to any theory, it is believed that the elevated and yet unknown anti-tumor activity is the result of the combination of the T cells, which are newly introduced into the patient by the administration of the T cell product of the invention, and the immune cells of the patients that has been subjected to immunosuppressant treatment, after being exposed to IL-2.

The combination with additional administrations of anti-IL-6Rand sTNF-αR can be used to prevent further hyper-inflammatory responses and to avert immune signatures that would lead to immune-exhaustion and which would have a negative impact on the interaction of the immune-cells and the tumor and/or the tumor cells directly.

Thus, according to an embodiment of the invention, each T cell administration is followed by separate administrations of IL-2, anti-IL-6-receptor antibody, and sTNF-αR.

The relatively low cell numbers in the T cell product used for administration, as compared to cell numbers administered in the methods of the prior art, can be sufficiently supported by a single dose of IL-2 post T cell administration. Accordingly, in an additional embodiment of the invention, IL-2 is administered as a single dose per T cell product administration.

IL-2 infusion can be performed up to one week after administration of the T cell product. IL-2 can be infused, for example, 8 hours after T cell administration, 12 hours after T cell administration, 16 hours after T cell administration, one day after T cell administration, two days after T cell administration, three days after T cell administration, four days after T cell administration, five days after T cell administration, six days after T cell administration, or seven days after T cell administration. In one particular embodiment of the invention, a single dose of IL-2 is infused 8 hours after the administration of the T cell product.

In an additional embodiment of the invention, IL-2 is administered in a concentration ranging from 20000 IU/kg to 720000 IU/kg. The concentration may be, for example, 20000 IU/kg, 40000 IU/kg, 60000 IU/kg, 80000 IU/kg, 100000 IU/kg, 120000 IU/kg, 140000 IU/kg, 160000 IU/kg, 180000 IU/kg, 200000 IU/kg, 220000 IU/kg, 240000 IU/kg, 260000 IU/kg, 280000 IU/kg, 300000 IU/kg, 320000 IU/kg, 340000 IU/kg, 360000 IU/kg, 380000 IU/kg, 400000 IU/kg, 420000 IU/kg, 440000 IU/kg, 460000 IU/kg, 480000 IU/kg, 500000 IU/kg, 520000 IU/kg, 540000 IU/kg, 560000 IU/kg, 580000 IU/kg, 600000 IU/kg, 620000 IU/kg, 640000 IU/kg, 660000 IU/kg, 680000 IU/kg, 700000 IU/kg, or 720000 IU/kg. Preferably, IL-2 is administered in a concentration ranging from 40000 IU/kg to 500000 IU/kg. More preferably, IL-2 is administered in a concentration ranging from 60000 IU/kg to 200000 IU/kg.

In one embodiment of the invention, IL-2 is administered in a concentration of about 60000 IU/kg. In another embodiment of the invention, IL-2 is administered in a concentration of about 120000 IU/kg. In a further embodiment of the invention, IL-2 is administered in a concentration of about 240000 IU/kg. In another embodiment of the invention, IL-2 is administered in a concentration of about 480000 IU/kg. In another embodiment of the invention, IL-2 is administered in a concentration of about 600000 IU/kg.

In another embodiment, anti-IL-6R is administered in a concentration of up to 10 mg/kg. The concentration may be, for example, 4 mg/kg, 5 mg/kg, 6 mg/kg, 7 mg/kg, 8 mg/kg, 9 mg/kg, or 10 mg/kg. Preferably, the concentration of anti-IL-6R is 4 mg/kg.

According to one embodiment of the invention, sTNF-αR is administered in absolute concentrations per subcutaneous administration of from 10 mg to 30 mg. The absolute concentrations may be, for example, 10 mg, 15 mg, 20 mg, 25 mg, or 30 mg. Preferably, sTNF-αR is administered in an absolute concentration of 25 mg per subcutaneous administration.

The compounds and cells used in the method can be delivered by administration routes known in the art. Suitable administrations routes are, for example, intravenous administration, subcutaneous administration, intra-arterial administration, intradermal administration, intrathecal administration.

The person skilled in the art is aware of different formulations of the compounds and cells to be administered in the method. As such, exemplary formulations may contain polyethylene glycol (PEG) or other substances supporting and/or facilitating the administration of the compounds or cells.

Moreover, the compounds administered can be obtained by well-known methods. Such methods may be, for example, production of proteins by recombinant means. Additionally, recombinant proteins can be produced in a variety of cell types that have been adapted to the production of recombinant proteins. Those cells can be transfected with the genetic construct of the respective protein to be produced by methods known in the art, e.g. retroviral, non-retroviral vectors, or CRISP-Cas9 based methods.

Preferably, the T cell product is administered via the intravenous route, intra-arterial route, intrathecal route, or intraperitoneal route, or directly into the tissue, or into the cerebrospinal fluid via a catheter. The immunosuppressant is preferably administered using the intravenous route as well. However, for primarily reducing and/or depleting Tregs the immunosuppressant can be taken orally. Moreover, the immunosuppressant can be administered intra-arterially, intrathecally, intraperitoneally, directly into the tissue, or into the cerebrospinal fluid via a catheter. The preferred administration route used for IL-2 administration is the intravenous route. However, IL-2 can be administered systemically or locally to the affected tissue or organ either in situ or intra-arterially. Subcutaneous administration of IL-2 is also possible, using a continuous infusion or a peak infusion with the dose provided within 20-30 min. Anti-IL-6R is preferably administered intravenously, intra-arterially, intrathecally, intraperitoneally, directly into the tissue, or into the cerebrospinal fluid via a catheter. For TNF-αR, subcutaneous administration is preferred, but the drug can also administered intra-arterially, intrathecally, intraperitoneally, directly into the tissue, or into the cerebrospinal fluid via a catheter.

In a further embodiment, anti-IL-6R is administered intravenously and TNF-αR is administered subcutaneously. Both administration routes represent the most suitable routes for the respective compound.

Accordingly, in one embodiment of the invention, the T cell product, the immunosuppressant, IL-2, and anti-IL-6R are administered via intravenous administration and TNF-αR is administered subcutaneously.

Cell Product for Treatment

In a second aspect, the invention provides a T cell product for use in the method of treating a tumor disease according to the first aspect of the invention. Accordingly, the T cell product for use is suitable for each of the embodiments relating to a method of treating a tumor disease according to the first aspect of the invention.

The T cell product according to the invention may be obtained from T cells or genetically engeneered T cells. According to one embodiment the T cell product is obtained from T cells and not from genetically engineered T cells. According to a further preferred embodiment, the T cell product is not obtained from CAR T cells.

The T cell product according to the second aspect of the invention exhibit anti-tumor activity. Anti-tumor activity can be assessed by methods known in the art as illustrated in examples 3 and 4 as presented herein. For example, the T cell product can be phenotyped and sorted for known cytotoxic T cells by using FACS as it is shown in example 3. T cells for which cytotoxic potential has been assigned in the invention are for example CD4+, CD8+, and/or CD107a+.

Another method to analyze anti-tumor activity is the measurement of IFN-γ production in the T cell product (see example 4.1). In the invention, a threshold is set at 200 pg/10⁵ cells/24 h upon 30 ng OKT3 stimulation. T cell products exhibiting an IFN-γ production above said threshold are assigned to a high anti-tumor activity. Anti-tumor activity can also be assigned to T cell products by the assessment of the specific cytotoxicity using a standard chromium-51 release assay as shown in example 4.2.

Preferably, the T cell product is obtained by expansion of T cells in the presence of IL-2, IL-15, and IL-21. According to a further embodiment of the invention, the concentration of IL-2 in the liquid composition is in the range of from 10 to 6000 U/ml. The International Unit (U) is the standard measure for an amount or IL-2. It is determined by its ability to induce the proliferation of CTLL-2 cells. The concentration of IL-2 is preferably in the range from 500 to 2000 U/ml. More preferably, the concentration of IL-2 is in the range from 800 to 1100 U/ml. According to one embodiment the concentration of IL-15 is in the range of 0.1 to 100 ng/ml. preferably, the concentration of IL-15 is in the range from 2 to 50 ng/ml, more preferably in the range from 5 to 20 ng/ml. The most preferred concentration is about 10 ng/ml. In a further embodiment of the invention, the concentration of IL-21 is in the range from 0.1 ng/ml, preferably in the range from 2 to 50 ng/ml, more preferably in the range from 5 to 20 ng/ml.

IL-2/IL-15/IL-21-expanded T cells used in the above method represent a highly effective approach to treat patients with a tumor disease. This is because the presence of IL-2, IL-15, and IL-21 during T cell expansion does not promote Treg outgrowth. Thus, in a further embodiment of the invention, the T cell product is manufactured by clonal expansion of autologous T cells of the patient in the presence of a cytokine cocktail comprising IL-2, IL-15 and IL-21, wherein the T cells are preferably isolated from a body sample selected from primary tumor, metastasis, or peripheral blood.

The body sample can be taken from any part of the body that contains T cells. Examples of body samples are primary tumor tissue, metastasis, and peripheral blood, e.g. PBMCs. As shown in the examples, the tumor can be successfully treated by the method according to the invention comprising administrations of a TIL cell product. Accordingly, in a preferred embodiment, the T cell product is a TIL cell product. According to further preferred embodiment the TIL cell product is obtained from GM TIL.

Methods for obtaining T cells are known in the art. For example, T cells can be isolated during surgical interventions such as biopsies (see example 1). T cells can also be isolated by aspiration of single cells from tissues and/or organs.

T cells can be expanded in the presence of IL-2, IL-15, and IL-21 directly after isolation from the body sample to save time until the resulting T cell product can be administered. Moreover, it is also possible to store the freshly isolated T cells or the T cell product obtained from a previous expansion until use, e.g. by freezing. The inventors found that an already obtained T cell product can be stored and re-expanded in the presence of IL-2, IL-15, and IL-21 and that this further expanded T cell product often exhibit altered anti-tumor activity.

In one embodiment of the invention, the anti-tumor activity of the T cell product in one administration is higher than the anti-tumor activity of the T cell product of the preceding administration. This has the advantage that if the patient's condition is good and the first T cell administration was tolerated well, the second T cell administration using a T cell product that exhibit higher anti-tumor activity may be sufficient to eliminate the tumor.

In another embodiment of the invention, the anti-tumor activity of the T cell product in one administration is lower than the anti-tumor activity of the T cell product of the preceding administration. This has the advantage that if the first administration was not tolerated well, the treatment has not to be stopped until the patient's conditions recovers, instead the therapy can be continued.

When the preceding step is a lymphodepletion and the preceded step is a T cell administration, the term “is preceded by” refers to a time point of 1 or 2 days before the T cell administration or a time interval of 1-2 days between lymphodepletion and T cell administration. If both involved steps are T cell administrations, the terms “preceding”, “preceded”, “is preceded by” refer to a time interval of from 1 week to several weeks or month between the two administrations. The exact time point for the second administration will be determine based on the clinical data of the patient. Importantly in this regard is that between both T cell administrations no additional T cell administration is performed.

Kit for Use in Treatment

The method described herein relies on high quality components and is a highly regulated process. In order to achieve best results and to facilitate preparatory actions for the user, the invention provides, in a third aspect, a kit for use in the method according to the first aspect of the invention.

Accordingly, in one embodiment, the kit for use comprises IL-2, IL-15, IL-21, anti-IL6-receptor antibody, sTNF-αR, and optionally at least one of a component that stimulates the TCR, in particular OKT3, costimulatory molecules, and feeder cells. In a further embodiment, the kit for use comprises all of these components.

The invention is further defined by the following examples.

### EXAMPLES

### Example 1—Isolation and Expansion of TIL and from GBM Patients

TIL were isolated from the GBM biopsy, cultured in medium containing IL-2, IL-15 and IL-21 (the cytokines may be, for example, obtained from Miltenyi, Bergisch Gladbach, Germany) first in 24-well plates in Cellgro medium (Cell Genix GmbH, Heidelberg, Germany) supplemented with human serum (10%), OKT3 (anti-human CD3 antibody, which may be, for example, obtained from Miltenyi) and allogeneic, 55-Gy irradiated feeder cells added on day 3 (1×10⁶ cells), followed by rapid expansion using OKT3 (30 μg/mL) and allogeneic, 55-Gy irradiated feeder cells. GMP-scale production of TIL for clinical use was carried out by Zellwerk GmbH (Berlin, Germany) using the ISO 13485-certified close perfusion bioreactor cell cultivation platform for advanced therapeutic medicinal products (ATMPs)¹⁷.

### Example 2—Method of Treatment and Tumor Progression

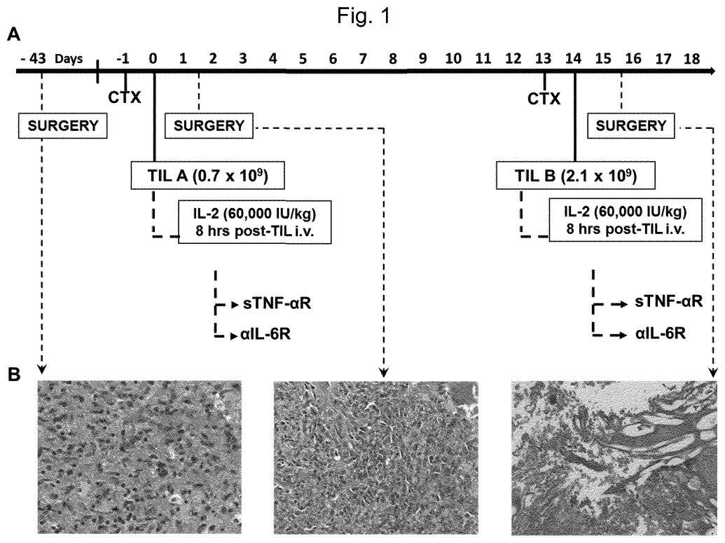

An overview of a representative treatment and the respective tumor progression is provided in FIG. 1. One day prior to TIL transfer the patient received cyclophosphamide dosed at 60 mg/kg. The next day, 0.7×10⁹ TIL (TIL-A) were administered by the intravenous route (i.v.) within 45 min. The TIL infusion was supported with a single dose of IL-2 (60,000 IU/kg, i.v.) administered 8 hours later as can be seen in FIG. 1, combined with infusion of anti-IL-6 receptor antibody (αIL-6R) and soluble tumor necrosis factor receptor (sTNR-αR) 24 hours later to prevent further cytokine toxemia. The patient was closely monitored for adverse events (AEs) and clinical development by MRI or CT according to immunotherapy Response Assessment in Neuro-Oncology (iRANO) recommendations¹⁴. The second TIL treatment was administered on day 14 with 2.1×10⁹ TIL (TIL-B) with cyclophosphamide treatment on day 0 and IL-2 administration 8 h post TIL in combination with infusions of αIL-6R and sTNR-αR as mentioned above.

The preparatory cyclophosphamide regimen used in the present study (a single dose of 60 mg/kg) differs significantly from that employed in known TIL studies, where the same dose of cyclophosphamide is used twice on days 1 and 2, followed by fludarabine at 25 mg/m² on days 3-7 preceding TIL infusions.

The conditioning regimen with a single cyclophosphamide dose given on day −1 mediated mild lymphopenia and moderate neutropenia but did not cause complete lymphodepletion in the patient. Based on the convincing clinical development showing a massive necrosis of the GBM tissue, it can be assumed that suppressive circulating Tregs were effectively reduced by the conditioning regimen¹⁸⁻²⁹.

During therapy, the patient was continuously monitored and tumor tissue was analyzed at different time points of the therapy. Therefore, diffusion-weighted magnetic resonance imaging (DWI-MRI) with apparent diffusion coefficient (ADC) or computed tomography (CT) with enhancement (following contrast agent administration) was used to gauge the radiological follow-up prior and after TIL therapy.

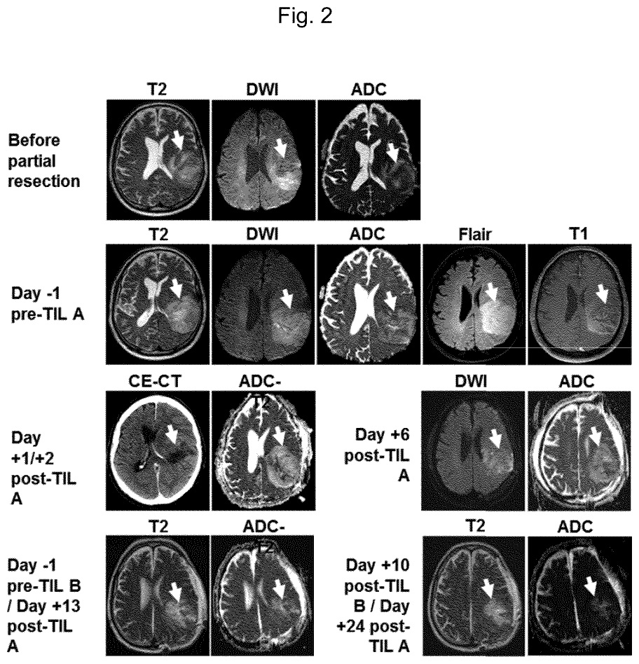

As shown in FIG. 2, the patient's first MRI at 6 weeks before first TIL (TIL-A) infusion (T2, before partial resection) showed a tumor with a cystic temporal component surrounded by a solid mass extending into the left parietal region. The mass showed initial signs of a midline shift to the right without herniation. DWI and ADC sequences showed diffusion restriction, with dense packing of cells representing a high degree of malignancy. The next MRI at day −1 pre-TIL-A infusion showed a massive progression of the solid lesion in all sequences (T2, DWI, ADC, Flair, T1 after contrast agent administration). The midline shift to the right revealed temporal herniation risk, with massive diffusion restriction of the solid lesion (DWI and ADC) with the tumor reaching into the mesencephalon. At day +1 after TIL-A application, a CT scan was performed, instead of an MRI. In the contrast-enhanced CT scan of the brain, a central necrotic lesion juxtaposed to the hemicraniectomy within the left parietal portion was visible, with reduced enhancement in the solid tumor. Accordingly, the MRI on day +2 showed T2 shine through in the ADC sequence, representing vasogenic edema instead of diffusion restriction. This was confirmed by DWI and ADC on day+6 after TIL-A infusion. The MRI on day +13 (post-TIL-A) showed a shrunken solid tumor dominated by a central necrotic portion (T2 sequence), confirmed by the high signal intensity in ADC representing T2 shine through. An MRI performed 10 days post-TIL-B infusion showed a dead cell mass in the solid tumor (T2 image). Due to brain compression symptoms after TIL-A and TIL-B transfer, surgical decompression was performed repeatedly. Biopsies of the tumor show complete necrotic tissue transformation as illustrated in the right image in FIG. 1B.

As a result, by using the method according to the invention it was possible to completely eliminate the patient's brain tumor.

### Example 3—Phenotyping of T Cells in the TIL Cell Product

Tumor reactivity of cells can be determined by phenotyping the cells within a TIL cell product, i.e. determining the cell composition in the TIL cell product. To perform such a definition of the cell composition in the TIL cell product the following methods were performed.

3.1 Methods

3.1.1 Flow Cytometric Analyses

Flow cytometry was performed to evaluate the phenotype, phorbol-myristate-acetate (PMA)-driven CD107a induction and Treg enumeration prior to TIL infusion.

3.1.2 T Cell Phenotype

1×10⁶ TIL were stained with the following antibodies: anti-human CD3 PE-Cy7 (BD Biosciences, Catalog Number: 563423), anti-human CD4 V450 (BD Biosciences, Catalog Number: 56345) and anti-human CD8a APC-Cy7 (BD Biosciences, Catalog Number: 557834). Acquisition of events was performed using a BD FACS Canto II flow cytometer (BD Biosciences, Stockholm, Sweden).

3.1.3 CD107a Induction

1×10⁶ TIL were incubated in RPMI medium (Gibco, Catalog Number: 61870-010) supplemented with 10% fetal bovine serum (FBS, Gibco, 10500-056), penicillin and streptomycin (Gibco, Catalog Number: 15140122), and 100 ng/ml phorbol 12-myristate 13-acetate (PMA, Sigma-Aldrich, Catalog Number: P8139) for 2 hours at 37° C. with 5% CO₂. The anti-human CD107a PE antibody (BD Biosciences, Catalog Number: 555801) and 4 μl of BD GolgiStop solution (BD Biosciences, Catalog Number: 554724) were also added to the cells during the incubation period in order to capture surface-bound CD107a molecules while halting their internalization. The cells were then washed and stained with the anti-human CD3 PE-Cy7, anti-human CD4 V450 and anti-human CD8a APC-Cy7 antibodies used in the T cell phenotype assay. The stained cells were washed once again and acquired on a BD FACS Canto II flow cytometer. Assays were performed in triplicates and control cells (IL-15, IL-2—activated T cells) were included to assure quality control.

3.1.4 Regulatory T Cells (Tregs)

1×10⁶ TIL were stained with the following antibodies: anti-human CD3 PE (BD Biosciences, Catalog Number: 555333), anti-human CD4 V450 (BD Biosciences, Catalog Number: 56345), anti-human CD8a APC-Cy7 (BD Biosciences, Catalog Number: 557834), anti-human CD25 PE-Cy7 (BD Biosciences, Catalog Number: 335824) and anti-human CD127 APC (Beckman Coulter, Catalog Number: B42026). After washing, cells were treated with the TrueNuclear Transcription factor buffer (BioLegend, Catalog Number: 424401), followed by staining with anti-human FoxP3 Alexa 488 (BD Biosciences, Catalog Number: 560047). The cells were incubated for up to an hour, washed and acquired on a BD FACS Canto II flow cytometer (BD Biosciences, Stockholm, Sweden). Assays were performed in triplicates and PBMCs from a healthy donor showing 2% of Treg, defined as CD3+CD4+, CD25high, IL-7Ra (CD127)−, were used as positive control cells for immunostaining.

3.1.5 TCR Vβ Repertoire

TCR Vβ repertoire in the TIL products was determined using the 10 Beta Mark TCR Vβ Repertoire Kit (Beckman Coulter, Catalog Number: IM3497) in the presence of co-staining with the following antibodies: anti-human CD3 PE-Cy7 (BD Biosciences, Catalog Number: 563423), anti-human CD4 Krome Orange (Beckman coulter, Catalog Number: A96417) and anti-human CD8a APC-Cy7 (BD Biosciences, Catalog Number: 557834). The stained cells were acquired on a BD Fortessa flow cytometer (BD Biosciences, Stockholm, Sweden). Data from flow cytometric acquisitions were analyzed using FlowJo software (FlowJo LLC, Oregon). The kit allows the coverage of approximately 70% of the TCR VB usage in humans.

3.2 Results

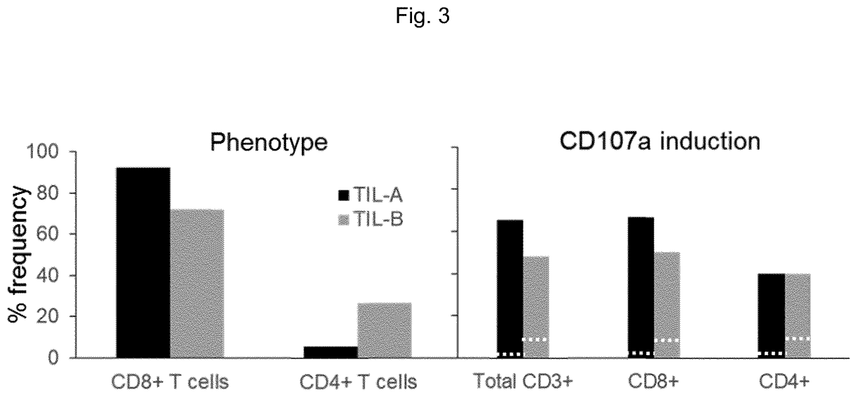

Flow cytometry phenotype analysis, as shown in FIG. 3, revealed approximately 90% and 70% CD8+ TIL in the first (TIL-A) and second (TIL-B) infusion products, respectively (left panel). CD4+ T cells increased from 5.4% in TIL-A to 26.4% in TIL-B (5-fold increase). 65% of the TIL, mostly CD8+ T cells, were CD107a+, exhibiting cytotoxic potential (right panel). CD25hi CD127− FoxP3+ regulatory CD4+ T cells (Tregs) were found negative (0.03%) in both TIL preparations.

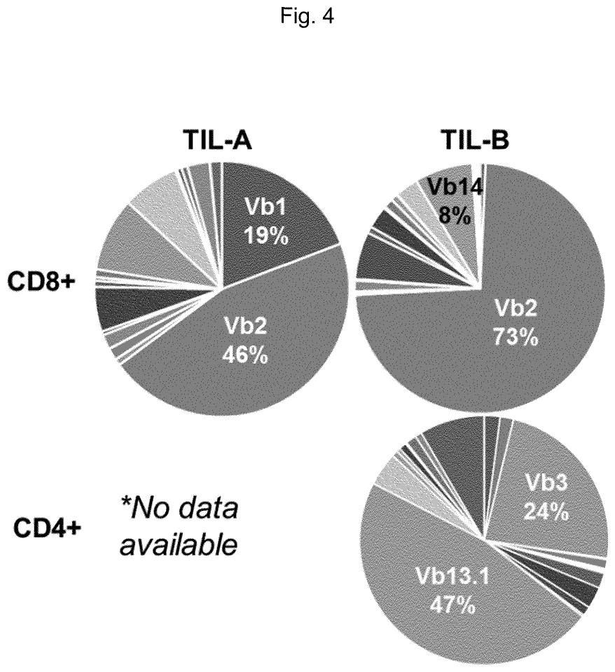

TCR Vβ flow cytometric analysis, as depicted in FIG. 4, showed that the TCR Vβ2 family represented approximately 46% and 73% of CD8+ T cells in TIL-A and TIL-B, respectively. The TCR Vβ1 family represented 19% of CD8+ T cells in TIL-A, while 8% of CD8+ T cells belonged to the Vβ14 family in TIL-B. CD4+ T cells in TIL-B were composed or 24% Vβ3 and 47% Vβ13.1 TCR families, respectively.

As shown, both TIL cell products comprise a vast amount of cell types to which anti-tumor activity can be assigned and thus may perform well in the method according to the invention. In order to determine the anti-tumor activity of the TIL cell product further assays can be performed.

### Example 4—Analyzing Anti-Tumor Activity

To test the ability of the TIL cell products for targeting and counteract tumor cells standard methods were performed to determine the level of anti-tumor activity.

4.1 Methods

4.1.1 IFN-γ Production

IFN-γ production was tested by stimulating TIL cell products with OKT3 for 24 hours followed by cytokine quantification in the culture supernatant by enzyme-linked immunosorbent assay (ELISA). Results are expressed as IFN-γ production pg/1.0×10⁵T cells/24 hours.

4.1.2 Chromium-51 Release Assay

Specific cytotoxicity was determined in standard chromium-51 (Cr⁵¹) release assays as previously described²¹. Briefly, autologous or control tumor cell lines (‘target cells’, T) were labeled with 100 μCi Na₂ ⁵¹CrO₄ for 2 hours. 1000 target cells were then incubated in V-bottom microwell plates with TIL (‘effector cells’, E) at different E:T ratios for 4 h at 37° C. Chromium-51 release was measured in the supernatant and specific cytotoxic activity was calculated by the standard method. For the cold target inhibition assays, a titration of the cold to hot tumor cells was done in the presence of 90:1 TIL. Following this, the TIL were pre-incubated at different ratios to the target cells with unlabeled autologous tumor cells as competitors at a ratio of 90:1 (cold:hot target) to block non-specific reactivity.

4.2 Results

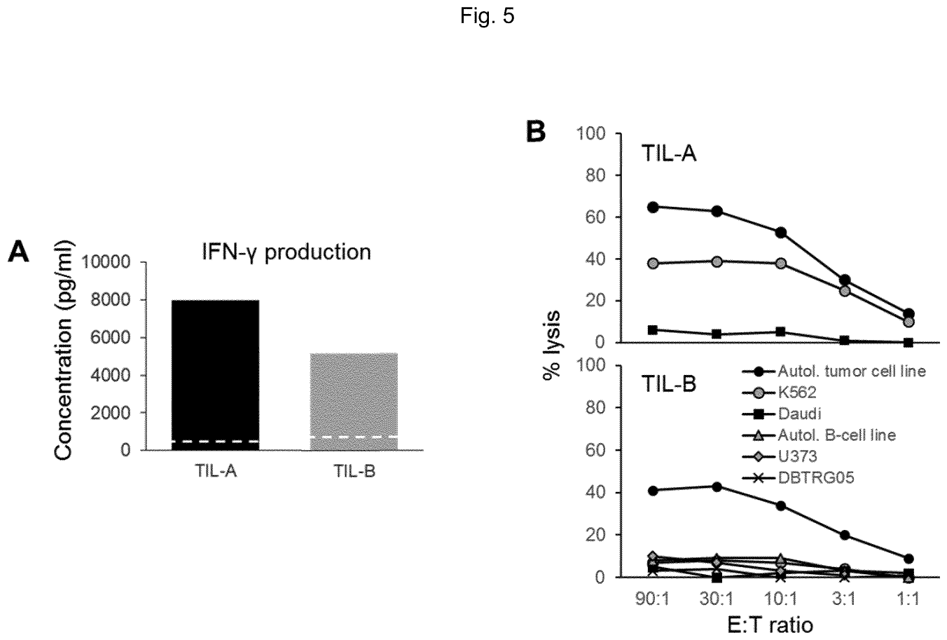

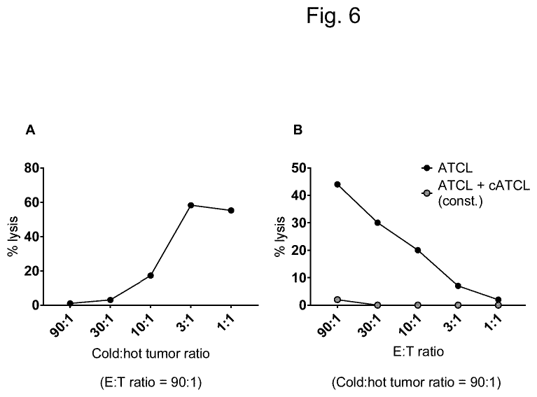

As shown in FIG. 5A, after a 24-hour stimulation of 1×10⁵ T cells with OKT3, IFN-γ production reached approximately 8000 pg/10⁵ T cells/24 hrs in TIL-A and 5000 pg/10⁵ T cells/24 hrs in TIL-B, respectively. Both TIL preparations specifically lysed the autologous GBM cell line in a dose-dependent manner, but not the autologous EBV-transformed B-cell line which can be seen in FIGS. 5B and 6.

The TIL cell products obtained from the expansion as described in example 2, exhibit good anti-tumor activity and can be applied in the method according to the invention.

### REFERENCES

1. Rosenberg S A, Restifo N P. Adoptive cell transfer as personalized immunotherapy for human cancer. Science 2015; 348:62-8.
2. Tran E, Robbins P F, Lu Y C, et al. T-Cell Transfer Therapy Targeting Mutant KRAS in Cancer. The New England journal of medicine 2016; 375:2255-62.
3. Andersen R, Donia M, Westergaard M C, Pedersen M, Hansen M, Svane I M. Tumor infiltrating lymphocyte therapy for ovarian cancer and renal cell carcinoma. Human vaccines & immunotherapeutics 2015; 11:2790-5.
4. Davis M E. Glioblastoma: Overview of Disease and Treatment. Clin J Oncol Nurs 2016; 20:S2-8.
5. Belhadj Z, Zhan C, Ying M, et al. Multifunctional targeted liposomal drug delivery for efficient glioblastoma treatment. Oncotarget 2017; 8:66889-900.
6. Zheng S, Cheng Y, Teng Y, et al. Application of luteolin nanomicelles anti-glioma effect with improvement in vitro and in vivo. Oncotarget 2017; 8:61146-62.
7. Kast R E, Skuli N, Karpel-Massler G, Frosina G, Ryken T, Halatsch M E. Blocking epithelial-to-mesenchymal transition in glioblastoma with a sextet of repurposed drugs: the EIS regimen. Oncotarget 2017; 8:60727-49.
8. Ferber S, Tiram G, Sousa-Herves A, et al. Co-targeting the tumor endothelium and P-selectin-expressing glioblastoma cells leads to a remarkable therapeutic outcome. eLife 2017; 6.
9. Rajora M A, Ding L, Valic M, et al. Tailored theranostic apolipoprotein E3 porphyrin-lipid nanoparticles target glioblastoma. Chem Sci 2017; 8:5371-84.
10. Quattrocchi K B, Miller C H, Cush S, et al. Pilot study of local autologous tumor infiltrating lymphocytes for the treatment of recurrent malignant gliomas. Journal of neuro-oncology 1999; 45:141-57.
11. Brown C E, Alizadeh D, Starr R, et al. Regression of Glioblastoma after Chimeric Antigen Receptor T-Cell Therapy. The New England journal of medicine 2016; 375:2561-9.
12. Liu Z, Meng Q, Bartek J, Jr., et al. Tumor-infiltrating lymphocytes (TILs) from patients with glioma. Oncoimmunology 2017; 6:e1252894.
13. Liu Z, Rao M, Poiret T, et al. Mesothelin as a novel biomarker and immunotherapeutic target in human glioblastoma. Oncotarget 2017; 8:80208-22.
14. Zanetti M. Tapping CD4 T cells for cancer immunotherapy: the choice of personalized genomics. Journal of immunology 2015; 194:2049-56.
15. Tran E, Ahmadzadeh M, Lu Y-C, et al. Immunogeneicity of somatic mutations in human gastrointestinal cancers, Science 2015, 10:1126
16. Tran E, Turcotte S, Gros A, et al. Cancer immunotherapy based on mutation-specific CD4+T-cells in a patient with epithelial cancer. Science 2014, Vol 344, 10.1126
17. Pörtner R, Parida S, Schaffer C, Hoffmeister H. Landscape of Manufacturing Process of ATMP Cell Therapy Products for Unmet Clinical Needs. Stem Cells and Tissue Engineering 2018.
18. Greten T F, Ormandy L A, Fikuart A, et al. Low-dose cyclophosphamide treatment impairs regulatory T cells and unmasks AFP-specific CD4+ T-cell responses in patients with advanced HCC. Journal of immunotherapy 2010; 33:211-8.
19. Madondo M T, Quinn M, Plebanski M. Low dose cyclophosphamide: Mechanisms of T cell modulation. Cancer treatment reviews 2016; 42:3-9.
20. Scurr M, Pembroke T, Bloom A, et al. Low-Dose Cyclophosphamide Induces Antitumor T-Cell Responses, which Associate with Survival in Metastatic Colorectal Cancer. Clinical cancer research: an official journal of the American Association for Cancer Research 2017; 23:6771-80.
21. Knuth A, Danowski B, Oettgen H F, Old L J. T-cell-mediated cytotoxicity against autologous malignant melanoma: analysis with interleukin 2-dependent T-cell cultures. Proceedings of the National Academy of Sciences of the United States of America 1984; 81:3511-5.
22. Sharpe M, Mount N. Genetically modified T cells in cancer therapy: opportunities and challenges. Disease Models & Mechanisms 2015; 8:337-350

1. A T cell product for use in a method of treating a tumor disease, wherein the method comprises one or more administrations of a T cell product, wherein at least one administration is preceded by a lymphodepletion, wherein said lymphodepletion comprises less than two immunosuppressant treatments.

2. The T cell product for use according to claim 1, wherein the method comprises at least two administrations of a T cell product.

3. The T cell product for use according to claim 1 or claim 2, wherein the content of regulatory T cells (Tregs) within the T cell product is below 2.5%, preferably below 1.5%, more preferably below 0.5%, most preferably below 0.1%.

4. The T cell product for use according to any one of claims 1 to 3, wherein the tumor disease is selected from brain cancer, pancreas cancer, hematopoietic tumors, tumors derived from the neural crest, and tumors of epithelial or mesenchymal origin.

5. The T cell product for use according to any one of claims 1 to 4, wherein the tumor disease is brain cancer, preferably the tumor disease is an astrocytoma, more preferably the tumor disease is glioblastoma multiforme (GBM).

6. The T cell product for use according to any one of claims 1 to 5, wherein each administration is preceded by a lymphodepletion, wherein each lymphodepletion comprises less than two immunosuppressant treatments, preferably each lymphodepletion comprises one immunosuppressant treatment.

7. The T cell product for use according to any one of claims 1 to 6, wherein the immunosuppressant is a cytostatic drug, preferably the immunosuppressant is selected from the group consisting of cyclophosphamide, azathioprine, methotrexate, and rapamycin.

8. The T cell product for use according to any one of claims 2 to 7, wherein the number of cells in the T cell product in one administration is higher than the number of cells in the T cell product of the preceding administration.

9. The T cell product for use according to any one of claims 2 to 8, wherein the number of cells in the T cell product in one administration is lower than the number of cells in the T cell product of the preceding administration.

10. The T cell product for use according to any one of claims 1 to 9, wherein the T cell product comprises a cell number of from 10⁸ to 10¹¹ cells, preferably from 10⁸ to 10¹⁰ cells, more preferably from 10⁸ to 10⁹ cells, and most preferably about 10⁸ cells.

11. The T cell product for use according to any one of claims 1 to 10, wherein the total concentration of the immunosuppressant in each lymphodepletion is up to 80 mg/kg, preferably up to 75 mg/kg, more preferably up to 70 mg/kg, and most preferably up to 65 mg/kg.

12. The T cell product for use according to any one of claims 1 to 11, wherein the concentration of the immunosuppressant in each treatment is in a range of from about 5 mg/kg to about 80 mg/kg, preferably from about 10 mg/kg to about 75 mg/kg, more preferably from about 15 mg/kg to about 70 mg/kg, most preferably from about 20 mg/kg to about 65 mg/kg.

13. The T cell product for use according to any one of claims 1 to 12, wherein each administration is followed by separate administrations of IL-2, anti-IL-6-receptor antibody, and soluble tumor necrosis factor receptors (sTNF-αR).

14. The T cell product for use according to claim 13, wherein IL-2 is administered as a single dose per T cell product administration.

15. The T cell product for use according to any one of claims 1 to 14, wherein the T cell product is manufactured by clonal expansion of autologous T cells of the patient in the presence of a cytokine cocktail comprising interleukin 2 (IL-2), interleukin 15 (IL-15) and interleukin 21 (IL-21), wherein the T cells are preferably isolated from a body sample selected from primary tumor, metastasis or peripheral blood.

16. The T cell product according to any of the preceding claims wherein the T cell is not manufactured from CAR-T cells, preferably the T cell product is not manufactured from genetically engineered T cells.

17. A Kit for use in a method of treating a tumor disease, wherein the method comprises one or more administrations of a T cell product, wherein at least one administration is preceded by a lymphodepletion, wherein said lymphodepletion comprises less than two immunosuppressant treatments.

18. A Kit for use according to claim 16, wherein the kit comprises IL-2, IL-15, IL-21, anti-IL6-receptor antibody, sTNF-αR, and optionally at least one of a component that stimulates the TCR, in particular OKT3, costimulatory molecules, and feeder cells.
